(19) United States
(12) Patent Application Publication
(10) Pub. No.: US 2014/0081385 A1
(43) Pub. Date: Mar. 20, 2014

(54) ARTERIAL IMPLANTS

(71) Applicant: Cytograft Tissue Engineering, Inc., Novato, CA (US)

(72) Inventors: Todd N. McAllister, San Anselmo, CA (US); Sergio A. Garrido, Buenos Aires (AR); Nicolas L'Heureux, San Rafael, CA (US)

(73) Assignee: Cytograft Tissue Engineering, Inc., Novato, CA (US)

(21) Appl. No.: 14/083,221

(22) Filed: Nov. 18, 2013

Related U.S. Application Data
(63) Continuation of application No. 12/485,898, filed on Jun. 16, 2009, now abandoned.
(60) Provisional application No. 61/132,085, filed on Jun. 16, 2008.

(51) Int. Cl. A61F 2/856

(52) U.S. Cl. CPC A61F 2/856
USPC 623/1.35

(57) ABSTRACT

The technology described herein generally relates to the field of tissue engineering and treatment of cardiovascular disease by endovascular repair. The technology more particularly relates to devices and methods to produce a tissue-based implant that can be used for abdominal aorta aneurysm, thoracic aorta aneurysm, or other cardiovascular repair.

### CROSS REFERENCE TO RELATED APPLICATIONS

This application is a continuation of U.S. patent application Ser. No. 12/485,898, filed Jun. 16, 2009 and now abandoned, which application claims priority from U.S. Provisional Application Ser. No. 61/132,085, filed on Jun. 16, 2008, both of which are incorporated herein by reference in their entireties.

### TECHNICAL FIELD

The technology described herein generally relates to the field of tissue engineering and treatment of cardiovascular and other disease by endovascular repair. The technology more particularly relates to devices and methods to produce and deploy a tissue-based implant that can be used for treating an abdominal aorta aneurysm, a thoracic aorta aneurysm or other cardiovascular repair.

### BACKGROUND

Abdominal aorta aneurysms (AAA) are defined as a dilation of the abdominal aorta, typically below the renal arteries, and with or without iliac involvement. This enlargement can progress until the point of rupture, which results in sudden death. In the U.S., approximately 75,000 patients are treated each year to repair abdominal aortic aneurysms. Historically, treatment has been performed in an ‘open’ procedure where a surgeon accesses the dilation through a peritoneal or retroperitoneal approach. This procedure is highly invasive, involves moving a number of vital organs including the intestines, and is associated with high mortality (˜5%), prolonged hospitalization, cardiac and renal complications, sexual dysfunction, and wound related complications such as hernia. Overall, AAA are the primary cause of death for approximately 15,000 patients each year, making AAA the 13th leading cause of death in the U.S.

Minimally invasive repair techniques have been reported, and are often referred to as endovascular abdominal aorta aneurysm repair (EVAR) (see, e.g., Parodi et al., Ann. Vasc. Surg., 5(6):491 (1991)). Today, more than half of all AAA repairs are performed using an endovascular approach. While design and delivery of endovascular devices is varied, all share the same basic approach: a synthetic graft (sometimes called an endograft or a stent graft) made of a material such as Dacron® or expanded poly-tetra fluroethylene (ePTFE) is manoeuvred into position by a catheter and caused to contact the interior of the arterial wall by deploying a balloon expandable or self expanding stent. The device is situated in the lumen of the aorta such that the endograft supports both arterial pressure and the arterial blood flow through the dilated portion of the aorta and into a healthy segment of the iliac artery (or arteries) (see FIG. 1).

There are several advantages to minimally invasive endovascular aneurysm repair techniques (e.g., shorter hospital stays, and a trend toward lower mortality rate), which have driven its rapid clinical adoption. Despite the popularity of the devices, however, the failure rate is approximately 15-20% within the first two years. The vast majority of failures are due to endoleaks, which is a leakage around or through the device. Other failure modes include embolization, infection, dissection of the aorta, etc. Most endoleaks occur due to inadequate anchoring of the device, which leads to problems such as relative motion, neck dilation or migration of the endovascular graft relative to the native aorta tissue. This relative movement and subsequent leakage often occurs in cases with well defined anatomical challenges, such as a short neck between the renal and iliac bifurcation, an angled neck, or a tortuous iliac artery. These anatomical challenges make it difficult to securely anchor the endograft, and as a result, blood can leak around the device, thus further pressurizing the dilated native aorta.

Efforts to reduce endoleakage have focused primarily on deployment strategies for metallic stents to more securely anchor the synthetic graft material to the native tissue. This approach, however, has met with little success, in that the non-compliant devices are anchored to an extremely elastic tissue which is dynamically loaded by both external (body movement) and internal forces (pulsatile blood flow). Additionally, the native tissue can remodel dynamically in response to these loads while neither the stent nor the synthetic material coating the stent can remodel. Though more recent devices use more flexible synthetic materials, the devices are still fundamentally unable to change the characteristics of the anchoring from the original implant configuration. As a result, endoleaks form from dislocations, fractures, translations, or migrations of the endovascular devices. Moreover, the fully synthetic materials described in previous devices often initiate chronic, mild inflammatory responses. These inflammatory responses can contribute to a variety of failure modes. Over the last 10 years or so, the primary focus of device manufacturers has therefore been on mechanical strategies to increase anchoring strength, using barbs, sutures, hooks, etc. Similarly, a significant effort has been put into appropriate sizing, and appropriate deployment strategies (such as optimizing degree of overinflation, placement of barbs, placement of endosutures, etc.) in an effort to optimize anchoring properties.

Recently, it has been proposed that proliferative factors such as fibroblast growth factor (FGF) impregnated into the graft material might more securely anchor the graft by enhancing cell migration and attachment to the endograft (see, e.g., Van der Bas et al., J. Vasc. Surg., 36(6):1237 (2002)). While this strategy helps to stabilize the aneurysm outside the device by increasing the fibrosis of the blood clot between the device and the diseased aorta, the approach is still fundamentally limited by the inherent differences in mechanical properties between the native tissue and the endograft, and there is a chronic mild inflammatory response associated with all biomaterials that may limit the incorporation of the graft material. Moreover, synthetic materials used to coat the stent are generally designed such that neither cells nor platelets can easily adhere to them, in order to prevent thrombosis in the lumen. Teflon, for example, is used as a stent graft material due to the advantageous characteristic that blood cells and platelets do not adhere to the Teflon surface disposed towards the lumen. Unfortunately, cells on the outside surface of the device, such as fibroblasts, similarly are weakly bonded to the material, leading to only a moderate anchoring strength between the device and the native vessel. Cell ingrowth and adhesion to the endograft is fundamentally limited with these materials, even with the addition of growth factors or paracrine agents.

Methods and devices are therefore desired that can be used, amongst other applications, to repair an AAA but without the problems of endo-leakage and anchoring that other approaches have suffered, and without inducing deleterious effects such as immune responses, in the subject.

The discussion of the background herein is included to explain the context of the inventions described herein. This is not to be taken as an admission that any of the material referred to was published, known, or part of the common general knowledge as at the priority date of any of the claims.

Throughout the description and claims of the specification the word “comprise” and variations thereof, such as “comprising” and “comprises”, is not intended to exclude other additives, components, integers or steps.

### SUMMARY

An artificial tissue construct, comprising: a trunk having a proximal end and a distal end; and two branches that connect to the distal end of the trunk; wherein each of the trunk and the branches comprises a tube of one or more tissue engineered sheets having a lumen.

An artificial tissue construct, comprising: a trunk having a proximal end and a distal end; a branch that connects to the distal end of the trunk; wherein each of the trunk and the branch comprises a tube of one or more tissue engineered sheets having a lumen; and an aperture on the trunk close to the distal end of the trunk and above the connection between the branch and the trunk.

A kit, comprising: a tissue construct of claim 2; and a second branch that is contralateral to, and separate from, the tissue construct; wherein the second branch comprises a tube of one or more tissue engineered sheets having a lumen.

A kit of artificial tissue, comprising: a trunk; and two branches that are separate from each other and from the trunk; wherein each of the trunk and the branches comprises a tube of one or more tissue engineered sheets having a lumen.

An implant, comprising: a trunk having a proximal end and a distal end; one or two branches that connect to the distal end of the trunk; wherein each of the trunk and the branches comprises a tube of tissue having a lumen; and one or more stents that are embedded within, mounted inside, of the sheets of one or more of the trunk and the one or two branches.

An implant, comprising: a trunk having a proximal end and a distal end; one or two branches that connect to the distal end of the trunk; a tube of tissue having a lumen disposed at the proximal end of the trunk; and one or more sleeves of synthetic material disposed over the remainder of the trunks and the branches.

An implant, comprising: a trunk having a proximal end and a distal end, wherein the trunk comprises a stent, and a tube of tissue disposed on an exterior surface of the stent at the proximal end of the trunk.

A method of making the tissue construct, comprising: seeding cells onto a cell culture substrate; growing the cells in vitro to form sheets; rolling the sheets into tubes to form the trunk and the one or two branches; and attaching the one or two branches to the distal end of the trunk.

A method of making an implant, comprising: seeding cells onto a cell culture substrate; growing the cells in vitro to form sheets; rolling the sheets into tubes to form the trunk and one or two branches; attaching the one or two branches to the distal end of the trunk; and mounting the one or more stents inside, outside, or both inside and outside of the sheets of one or more of the branches and trunk.

A method of making the implant, comprising: seeding cells onto a cell culture substrate; growing the cells in vitro to form sheets; expanding the one or more stents; rolling the sheets around the expanded stents; suturing the sheets into tubes to form the trunk and one or two branches; attaching the one or two branches to the distal end of the trunk; and collapsing the stents.

A method of deploying an implant in a subject, comprising: making an implant according methods described herein, wherein the attaching takes place inside the subject.

A method of treating a condition in a subject, the method comprising: replacing or reinforcing a portion of one or more contiguous blood vessels of the subject with the artificial tissue construct or the implant described herein.

Other features and advantages of the invention will be apparent from the following detailed description, and from the claims.

It should be noted that while anchoring limitations and subsequent migration are primarily associated with abdominal or thoracic aorta repair, there are several other cardiovascular repair devices that would be improved using the tissue wrapped stent grafts described herein.

### BRIEF DESCRIPTION OF THE DRAWINGS

FIG. 1 is a ePTFE-wrapped unimodular endograft for abdominal aorta aneurysm repair.

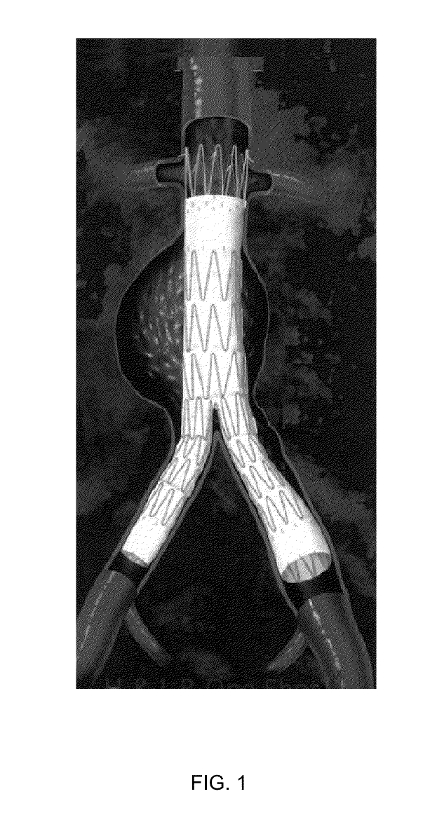

FIG. 2 is a unimodular device made from a tissue sheet rolled around a stent, and two sheet based tubes. The sheets are secured to the stent and to each other via sutures.

FIG. 3 shows a contiguous bifurcated tissue-engineered construct.

### DETAILED DESCRIPTION

The technology described herein is related to tissue-based methods and devices for blood vessel repair, for example, repair of an abdominal or thoracic aortic aneurysm. The origin of the damage that is in need of repair can be disease, or can be a trauma, aging, a birth or other genetic defect, or from a systematic injury. An embodiment of the technology could also be used for peripheral or coronary stenting.

As used herein, the term “tissue engineering” means the in vitro formation of tissue structures, such as those that are suitable for replacing or augmenting anatomical structures, from living tissue cells, where the structures are formed by the cells themselves under suitably employed culture or growth conditions. This can be accomplished by using the cells only to form the tissue, or it can be accomplished by seeding the cells into a scaffold material. Other ingredients, including non-naturally occurring ingredients, may be added to the culture milieu to facilitate the appropriate tissue growth. Tissue structures that can be grown by tissue engineering include, but are not limited to, sheets, ribbons, tubes, caps, and sacs.

A tissue structure may be made by tissue engineering or may be made by assembling pieces of tissue obtained from, e.g., a human subject or an animal.

A tissue construct, as used herein, means an article that is made from a tissue structure, in whole or in part.

Artificial means, as used herein, made by man, directly, or indirectly by a man-made machine or device.

In one embodiment, the technology herein comprises an implant having a tube of tissue disposed on the exterior surface of a stent. Such an implant has a proximal end, that would be situated in an upstream portion of a vessel such as an artery, and a distal end. The tube of tissue is typically covering at least the proximal end of the stent, though barbs may extend out beyond the tissue. The tube of tissue may be made from one or more tissue engineered sheets, wrapped around and joined to one another. The tube of tissue may also be made from tissues that have been harvested, e.g., from the subject in which the device is to be implanted, or from another subject, or from an animal. The tube of tissue is typically joined to the stent by methods described elsewhere herein. Such an implant may be suitably disposed in a region of the thoracic artery, the abdominal aorta, or another suitable artery or vessel. In securing, e.g., the implant in the thoracic aorta, a staple or suture may be placed from the outside. The implant may be fixed to the interior wall of the artery or lumen, when initially deployed, by methods described elsewhere herein. Cells suitable for making the tissue are also described elsewhere herein. Additional tissue layers may be lined on the interior of the stent.

In one embodiment, the technology herein utilizes an implant that is an artificial tissue construct, having a trunk with a proximal end and a distal end, and one or two branches that connect to the distal end of the trunk. The trunk and the branches are each made, in part, from a tube of one or more tissue engineered sheets of living or devitalized cells, and have a lumen. In some embodiments the tissue engineered sheets wrap around the inside, outside, or both inner and outer surfaces of a stent. In the case of a tissue sheet disposed on the inside surface of a stent, during expansion of the stent, the tissue sheet is pressed against the stent framework and allows cells from the sheet to contact the inner surface of the lumen in which the device is disposed. In the case of thoracic, coronary, or peripheral limb vascular repair, the device is typically comprised of a non-bifurcating trunk only. The stent can be made in whole or in part from a material such as a bioresorbable metal, one or more polymers, or one or more biological materials. Synthetic materials such as Dacron or ePTFE can be used either as a circumferential wrap or as a segmental wrap. The region adjacent to and including the proximal end of the trunk is referred to herein as the neck. Synthetic materials are of particular use in the distal regions of the device and/or on the distal portion of the trunk. Conversely tissue-engineered sheets are desirably situated on the exterior surface of the neck because this leads to considerably improved anchoring of the device, also referred to herein as “biological neck fixation” (BNF). Such an implant may also be referred to as an endograft or a stent graft device.

Note also that these endograft devices can also be assembled in vivo. By embedding the stent in certain body cavities or under the skin, a tissue scar can form around the device. This sheath, formed by the scarring or encapsulation response, has similar properties and similar functions to the tissue engineered sheet created in vitro and then wrapped around the stent.

The technology herein, particularly for AAA repair, utilizes delivery methods and anchoring techniques not described elsewhere. The cell-based approaches to EVAR described herein address the primary failure modes associated with existing endovascular devices by providing either or both of a mechanical and a cellular based fixation methodology. BNF provides for durable and secure fixation of an implant to the vessel that can grow and remodel in response to the local mechanical environment or adapt to growth/relative motion between the anchoring points in the native tissue and the implant. Once in place, the tubes of tissue engineered sheets support blood flow (and transluminal pressure gradients) through or around the diseased (or damaged) portion of blood vessel without further dilation/rupture of the native tissue in the region. The tissue that forms the mechanical support for this implant can then become incorporated into the surrounding tissue and the native vessel over time, typically weeks to months, depending upon the cell types, and the degree of injury, thus providing a leak-tight seal that can grow, remodel and move with the native tissue.

The technology described herein has multiple key advantages over previously described approaches. In one respect, this technology herein provides a long-term fixation method that is based upon cellular adhesion/incorporation between the living host tissue and cell produced sheet (living, decellularized or divitalized). In another respect, the technology herein provides implants that not susceptible to endo-leaks.

Representative compositions of the devices, and of methods of making and using them are further described herein.

### Compositions

The devices described herein include a tissue engineered sheet or combination of tissue sheets that can be formed into a tube with an open lumen, or plurality of lumens, that can carry blood flow through a diseased blood vessel(s). The devices can either have more than one tubular portion joined together, i.e., can be bifurcated (“unimodular”), or can comprise two or three disjoint tubular portions, i.e., can be unbifurcated (“bimodular”, or “trimodular”).

In the bifurcated (unimodular) mode, the device comprises a trunk having a proximal end and a distal end, and two branches that connect to the distal end of the trunk. Each of the trunk and the branches comprises a tube of tissue, such as one or more tissue engineered sheets, having a lumen. In repairing an AAA, the trunk is disposed in the abdominal aorta, and one branch is disposed in an iliac arterial vessel, the other in the contra-iliac arterial vessel. The implant then adopts an inverted “Y” configuration when inserted.

In the bimodular mode, the device comprises a trunk having a proximal end and a distal end, a branch that connects to the distal end of the trunk, and an aperture on the trunk close to the distal end of the trunk and above the connection between the branch and the trunk. A second branch, which is contralateral to the first branch, is initially provided separate from the trunk. This second branch is connected to the aperture on the trunk close to the distal end of the trunk before or during the surgery that takes place to insert the device. Each of the trunk and the branch comprises a tube of tissue, such as one or more tissue engineered sheets, having a lumen.

In the trimodular mode, the device comprises a trunk having a proximal end and a distal end. The two branches are initially provided separate from each other and from the trunk. These two branches are connected to the distal end of the trunk before or during the surgery. Each of the trunk and the branch comprises a tube of tissue, such as one or more tissue engineered sheets, having a lumen.

The tubes of tissue, such as tissue engineered sheets as described herein, can be used for the entire device, i.e., the trunk and the two branches. The tubes of tissue can also be used for only parts of the device, for example, at the neck of the trunk, or at regions adjacent to and including the proximal or distal ends of one or both branches, or combinations of such configurations. In such instances, a synthetic material, such as ePTFE or Dacron®, can be used for the remainder of the trunks and the branches that are not covered in tissue.

A synthetic support sleeve can be added inside or outside the tubes of tissue of one or more of the trunk and the one or two branches. The tissue constructs can also be fenestrated to allow additional branching to feed side arteries such as the renal, mesenteric, or subclavian arteries.

A catheter-based delivery system can be used to deliver the device to the location of interest, and then to deploy and anchor the trunk and the one or two branches within the cardiovascular system of a subject.

In another embodiment, stents are lined with tubes of tissue, such as made from tissue engineered sheets. For example, the stents can be embedded within, mounted inside, mounted outside, or mounted both inside and outside of portions of the sheets of one or more of the trunk and the one or two branches. The tubes of tissue, such as tissue-engineered sheets, can be anchored to the stents by several methods, for example, suturing, or allowing the living sheets to adhere to the stent via tissue ingrowth. Alternatively, a tissue-engineered sheet can simply be wrapped around the stent. The stents can be placed at the ends of tubular sheets only, or can run the entire length of the tubular portion in question. Similarly, the stent can be segmented such that it overlaps only portions of the tissue. The ends of the stent can extend beyond the end of the tissue to provide increased anchoring strength via mechanical means, as applicable. The device can also include a way for attaching endosutures to increase anchoring strength.

The stents, as used in the devices herein, can be continuous or segmented. The stents can be balloon-expandable, self-expandable, collapsible and re-expandable, or adjustable. The stents or part of the stents can be resorbable, or comprise a series of barbs for facilitating anchoring to the interior of a lumen, or for securing a tube of tissue, such as a tissue engineered sheet thereto.

### Method of Making

Certain production methods for a tissue-based sheet, suitable for use with the devices and implants herein, have been previously described elsewhere (see, e.g., U.S. Pat. Nos. 7,112,218, 7,166,464, 7,504,258, and 6,503,273, and L'Heureux et al., FASEB J 12(1):47 (1998), all of which are incorporated herein by reference in their entireties). Using this approach, grafts with mechanical properties very similar to that of native arteries can be built without the addition of exogenous materials or synthetic scaffolds. Advantages of this approach include that the tissues made are compliant, are non-thrombogenic, are comprised of living cells so the prosthesis can grow/remodel with the patient, and, because they are completely human derived, initiate little or no immune responses. Methods to wrap or embed the entire length of expandable stents within sheets of tissue have been previously disclosed (U.S. Pat. No. 7,166,464). These methods may minimize thrombogenic and/or inflammatory mediated responses and provide an enabling platform for cell-produced anti-restenotic agents. The devices herein are not limited in their construction to those made with such methods, as other improvements, and variants thereof known by those skilled in the art may also be applicable. For example, other tissue-engineering routes to make a sheet that may be used include the use of porous materials, a tubular conduit, and a rolled sheet. In other approaches, a stent may be cast into a porous gel (polymer, hydrogel, collagen, etc.) and cells seeded into it. In still other embodiments, a tissue sleeve can be formed.

In outline, in a method suitable for making tissue-based sheets herein, cells are seeded onto a cell culture substrate and grown in vitro to form sheets. The sheets are rolled into tubes to form, separately, the trunk and the one or two branches. Alternatively, the cell culture substrate may incorporate a tubular structure, such as a removable mandrel, so that the sheets are grown directly in a tubular configuration, without requiring a separate rolling step. The tubular construct can also be grown by seeding cells onto the mandrel directly. The one or two branches, regardless of their method of construction, can be attached to the distal end of the trunk, or the trunk can be used alone as a non-bifurcating implant. Optionally, one or more stents are mounted inside, outside, or within the sheets of one or more of the branches and trunk. In another embodiment, stents are expanded and the sheets are rolled around the expanded stents and sutured into tubes to form the trunk and one or two branches. The one or two branches are attached to the distal end of the trunk, and the stents are collapsed to facilitate endovascular deployment. The branches can also be connected using glue, staples, sutures, or other techniques known in the field. The branches can also be matured in culture such that the bifurcation is ‘grown’. In still another embodiment, a unimodular tissue construct can be grown in one piece. In such embodiments, additional support for the joint regions can advantageously be applied.

In some embodiments, cells, such as fibroblasts, smooth muscle cells, bone marrow derived cells, circulating stem/precursor cells, endothelial cells, or other cells that can be directed into mesenchymal or structural cell lineages can be seeded onto a cell culture substrate and grown for prolonged periods of culture time in vitro to form a robust sheet. Typically this sheet production time would range between 2 and 16 weeks, such as 4 to 12 weeks, or 6 to 10 weeks, or 8 weeks. Sheets can be produced more rapidly if derived from an animal tissue or from cells seeded into an existing scaffold, rather than being required to culture an entire sheet. In some embodiments, the cells are not endothelial progenitor cells (EPC) because such cells do not have sufficient mechanical integrity to form manipulatable structures. The cells can be of autologous, allogeneic, or xenogeneic origin. The tissues can also be comprised of a combination of cell sources (such as an allogeneic or xenogeneic sheet seeded with autologous endothelial cells). The sheets can also utilize cells that have been genetically modified to express desired proteins, such as growth factors, angiogenic factors, therapeutic factors, or factors altering the mechanical properties of the sheets, the integration of the sheets into the surrounding tissue, the restenosis of the tissue, or the inflammatory responses of the tissue. The sheets can also utilize cells that have been genetically engineered to grow into tissue structures that have mechanical integrity, such as being manipulatable by hand or tool. Alternatively, sheets can be derived from human or animal tissues such as pericardium, peritoneum, or intestinal submucosa. The sheets can be all or partially living, devitalized, or decellularized. Combinations, such as tissue sheets that are then repopulated with a subject's own cells can also be used.

Once the sheet acquires sufficient strength such that it can be detached (and manipulated mechanically, e.g., onto a backing sheet) from the cell culture substrate and transferred onto the stent portion of the endograft device or a mandrel, it can be formed into a tubular structure with appropriate lumens and then anchored to the native tissue to re-route blood flow through or around diseased or otherwise damaged tissue. The tubes can be further matured in culture to fuse the sheets of each tube together. A protein or an adhesive agent can be added to the sheets prior to rolling the sheets into tubes. The sheets can be sewn together, either before or after mounting the sheets to the stent. The tubes can be tapered, bifurcating, or straight. The tubes can also have reinforcements or ribbed structures to assist with fixation in the artery, for example, by rolling the sheets with a soft rib of thicker tissue at both ends of the tubes, thereby increasing tube diameter and contact area at the ends of the tubes. They can also include devices or markers to limit twisting, misplacement, or migration during deployment. They can also be scalloped or shaped to increase elasticity and compliance. The tubes have an external diameter suitable for the intended use, for example, about 16-40 millimeters for the trunk, about 6-25 millimeters for each branch, in the case of AAA repair. For coronary and lower limb uses, non-bifurcating tubes with smaller diameters, such as 2-15 mm, can be used.

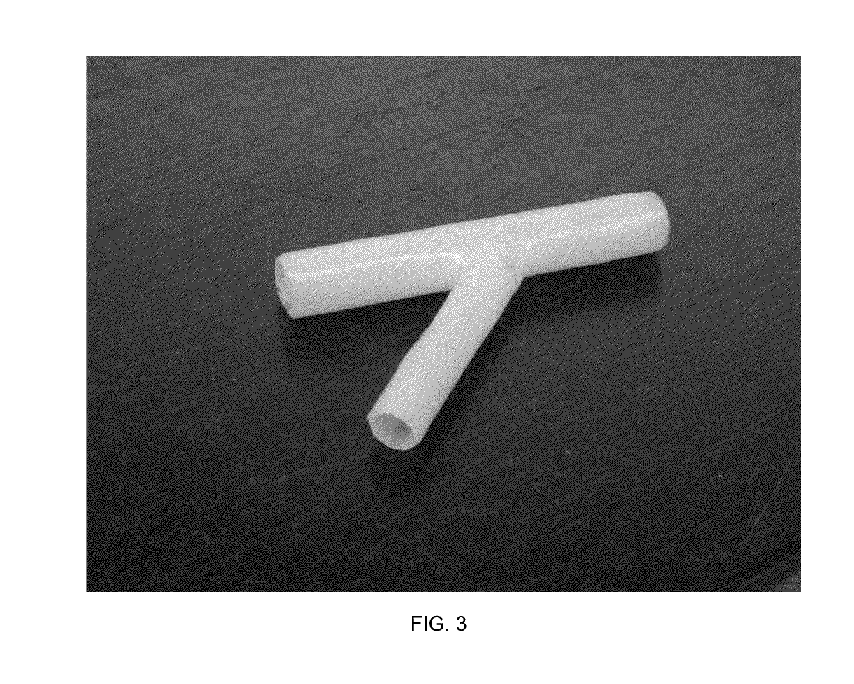

One or both of the branches can be attached to the trunk by several methods, for example, mechanical fixation, growing the trunk and one or more branches as a contiguous bifurcating graft (see, e.g., FIG. 3) or anastomosis.

The tubes can be delivered to the patient with or without structural stents. The stents, where used, can be continuous or segmented to allow customization. The sheets can be secured via sutures or other mechanical fixation (staples, etc.), chemical (e.g., glue), or via biological approaches such as biological glues or cellular adhesion. The stents can be completely embedded within the tissue or can be on the inner and/or outer layer of the sheet. The sheets can also be impregnated or coated with a paracrine factor such as heparin, a growth factor, an adhesion factor, or a pharmacological agent such as an anti-restenotic drug or protein. The device can also include a support sleeve made from a synthetic material, such as Dacron® or ePTFE, which is placed either within the roll of tissue or wrapped around the outside, or a portion thereof, as a sleeve. This support sleeve can help to provide increased short term strength which thereby decreases production times for the sheet of the overall device. The stents can protrude from the ends of the tissue sheet to increase mechanical anchoring without occluding side branches of the native vessel.

### Method of Using

The tissue-based devices described herein, with or without the stents, can be used to replace, re-line, or reinforce a portion of one or more contiguous blood vessels in a subject having a disease such as an abdominal aortic aneurysm, peripheral vascular disease, and coronary vascular disease. The devices can be used for an animal or a human. The devices can be delivered to a subject by several methods, for example, open surgical, endovascular, thoracoscopic, and laparoscopic procedures. The tubes of tissue on the exterior of the devices can be initially anchored to the native tissue by several methods, for example, sutures, staples and/or expandable stents.

### REPRESENTATIVE EMBODIMENTS

The following representative embodiments of the technology are presented to illustrate various aspects of construction, manufacture, and use in a manner which is not intended limit the scope of the technology described in the claims. It would be understood that where an aspect of construction, manufacture, or use is discussed in the context of one embodiment, such aspect could also be applied to some other embodiment, even though not explicitly delineated.

### A Unimodular Bifurcating Device Having Tissue Engineered Sheets

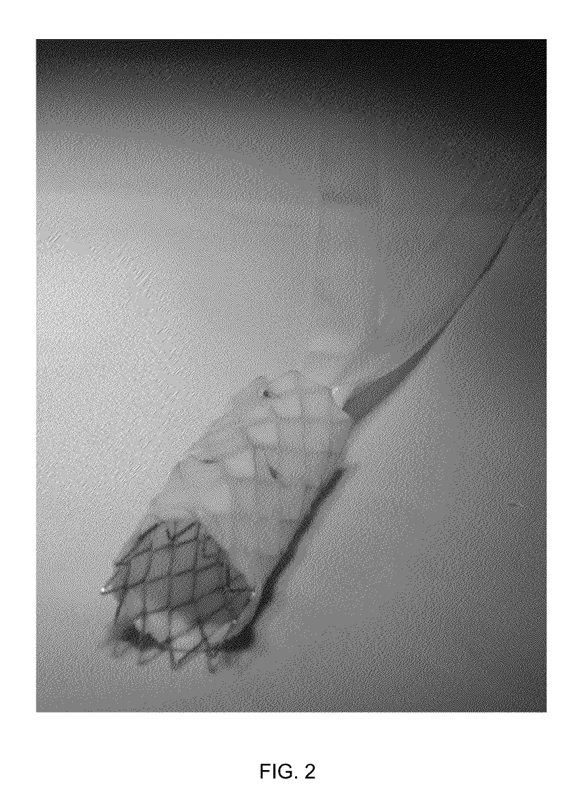

A unimodular bifurcating device is built by joining three rolled tubes of tissue as illustrated in FIG. 2. The main trunk (typically 18-38 mm in external diameter) is assembled by rolling one or more tissue engineered sheets into a tubular structure. An expandable stent, such as a balloon expandable stent (e.g., a Palmaz stent) can be used to initially anchor the main trunk to the proximal arterial region with or without suprarenal fixation. The Palmaz stent can be embedded within the tubes of tissue or can be mounted on the inside surface of the tubes of tissue. The stent can also protrude from the end of the rolled tissue. Alternatively, the tissue can be placed on the lumen of the stent. In each case, the stent is collapsed, and the tissue carefully collapsed with it. This allows the main trunk (along with the bifurcation branches) to be delivered via an endovascular approach as described elsewhere herein. By expanding the stent inside the rolled tube to a diameter slightly larger than the native artery (e.g., the aorta) (typically approximately 5% over native diameter), the proximal end of the endograft device can be anchored to the native tissue. This initial mechanical anchoring system is supplemented over time by the cellular activity/adhesion between the endograft tissue and the native tissue.

The biological fixation of the device to the native tissue can be enhanced by rolling the sheet with a soft rib or band of thicker tissue at the end to increase device diameter and contact area. The tissue-cell based adhesion is the basis for biological neck fixation as described elsewhere herein. In order to strengthen the rolled tube, the layers of the sheet can be connected together (sewn or glued together, for example), to limit unrolling and/or to prevent twisting or migration after implantation. The rolled sheets can also be matured for extended periods of time such that they fuse together in culture.

The rolled sheets can also be left unfused to increase the ability to expand and deploy the device. Two smaller tissue tubes (typically 7-20 mm in external diameter) are provided for the distal ends of the endograft. These tubes, again made by rolling a tissue sheet, are inserted into the iliac or femoral arteries. The branch tubes can be attached to the main trunk via sutures or other mechanical fixation, or can be grown as a contiguous bifurcating graft. As described elsewhere herein, other fixation techniques (gluing for example, can be envisaged). Examples of mechanical fixation include suturing, stenting (expanding a stent to compress the device against the native vessel wall), or stapling. As described elsewhere herein, the tubes can be strengthened by connecting the layers of the sheet together (also via mechanical means of fixation, or via cellular/protein binding). The branching tubes can also be made of a synthetic tube, since the requirement for anchoring strength is primarily at the proximal end, or neck, of the device. The proximal end of the bifurcation branches are secured to the distal end of the main trunk using standard anastomotic techniques (for example, Prolene® sutures). The anastomoses can be made either before the implantation or intra-operatively during the implant procedure. Similarly, the distal ends of the branches are sewn to the native iliac or femoral artery to provide a leak-tight anastomosis. This anastomosis is preferably made during an open procedure where both iliac or femoral arteries are exposed surgically. Alternatively, the anastomosis can be used using a mechanical device such as an expandable stent that can also be deployed via an endovascular approach.

The endograft device can be collapsed into a sheath and delivered into the abdominal aorta via the femoral or iliac artery via a catheter. A second catheter can be inserted from the contralateral femoral or iliac and advanced up to capture the contralateral branch of the device. Typically, these are multiple lumen catheters which allow the introduction of multiple devices within the original introducer catheter. By pulling back the second catheter, the contralateral branch is deployed in the contralateral iliac or femoral artery. The proximal end of the endograft device can then be located radiographically and secured by expanding the stent or deploying another mechanical anchoring device such as staples or barbs. Alternatively, twisting of the device after implantation can be prevented by stiffening the legs of the bifurcation against torsional rotation. The iliac or femoral branches are then cut to length and secured using open anastomotic techniques common to vascular surgery. Each branch requires two sealing procedures. In addition to the connection between the distal end of the endograft and the distal portion of the resected native iliac/femoral artery, the proximal portion of the native iliac/femoral must be sewn to the endograft to prevent leakage from collateral vessels.

There are several variations on the main principles of the tissue covered stent and biological neck fixation described herein that fall within the scope of the technology described herein: Clearly the size range (e.g., diameter and length, of trunk and branches) can vary dramatically to address a variety of human and non-human physiologies. There are also a wide variety of mechanical fixation techniques that could be employed. For example, the expandable stents could be used for both the main neck and the bifurcations. The stents could be self-expanding or balloon-expandable. In an important derivative of the technology, the stent could be resorbable or could be a series of simple barbs. Since within a few weeks the endograft will be incorporated into the native tissue, the actual components of the ‘stent’ can be minimized. There are also several variations for delivery and deployment that can be envisioned. The contralateral branch, for example, could be deployed by a separate wire captured from the contralateral approach. Cellular and protein components can also vary. The sheets can be seeded or combined with other cell types. Protein or chemical glues can be added to increase strength or adhesion within the sheet/roll. There are also several genetic variations that can be envisioned. Genetically modified cells to express growth factors, angiogenic factors, therapeutic factors, or factors that alter the mechanical properties of the sheets can also be envisioned.

### A Bimodular Device

In a bimodular device, the trunk and one branch, e.g., of the iliac, are built. Such a device can be deployed to treat AAA by using biological neck fixation—from tissues situated on the exterior surface of the device—at the proximal portion of the trunk, situated in the aorta, and an open anastomosis at the distal iliac/femoral artery as described in connection with the unimodular embodiments, hereinabove. The contralateral branch is then deployed separately via the contralateral native iliac/femoral. The separate branch is secured to the main trunk (or, in some embodiments, a stub or a short leg branching off the main trunk) via mechanical fixation devices such as by suturing, or attaching to a stent.

Several variations of manufacture, construction and use, as described in for the unimodular embodiments, can similarly be envisioned for a bimodular device.

### A Trimodular Device

The endograft can also be a trimodular device with a main trunk and separate legs to form the bifurcation. In a trimodular device, both branches of the graft are delivered separately. The branches of the endograft device are each separately secured as described in connection with the bimodular embodiments hereinabove.

Several variations of manufacture, construction and use, as described for the unimodular embodiments, can be envisioned for a trimodular device.

### An Aorto-Monoiliac/Femoral Device

In an aorto-monoiliac/femoral device, the contralateral limb of the endograft is eliminated entirely. The contralateral main iliac artery is occluded and flow to the contralateral limb is supplied via the femoral-femoral or iliac or iliac bypass.

### Delivery by Endovascular Approaches

The various devices described herein can be delivered via a totally endovascular approach, typically via the femoral artery in the case of treating an AAA. It should be noted that this unique delivery system can also be utilized to deliver other non-biological endograft devices.

### EXAMPLES

The technology is further described in the following examples, which do not limit the scope of the technology described in the claims.

### Example 1

### Unimodular Bifurcated Implant

A bifurcated implant was obtained by wrapping a tissue-engineered sheet around a Palmaz-balloon-expandable stent to form a main trunk. The ends of two tissue-engineered blood vessels (TEBV) were sewed together. The joined two-vessel assembly was sewed to one end (ultimately the distal end) of the main trunk. The sewing looks like a “FIG. 8” configuration. The stent was collapsed around a catheter, and the entire assembly was fed up the femoral artery. By coming in with a wire, it was possible to grab the other leg from the contra-lateral artery and pull it back down that artery. The stent was inflated at the proximal neck. It is not necessary to carry out an expansion of the branches in the iliac and contra-iliac (although it could be done). Instead, it is more practical to use a small incision in the iliac and sew in those branches.

### Example 2

### A Tissue-Coated Synthetic Implant

A tube of tissue is placed around the neck of a bifurcating device, to provide anchoring, when implanted in vivo. The underlying bifurcating device is made from a synthetic material such as Gore-Tex® or Dacron®.

### Other Embodiments

It is to be understood that while the technology has been described in conjunction with the detailed description thereof, the foregoing description is intended to illustrate and not limit the scope of the technology, which is defined by the scope of the appended claims. Other aspects, advantages, and modifications are within the scope of the following claims.

1. An artificial tissue construct, comprising:
a trunk having a proximal end and a distal end; and
two branches that connect to the distal end of the trunk;
wherein each of the trunk and the branches comprises a tube of one or more tissue engineered sheets having a lumen.

2. An artificial tissue construct, comprising:
a trunk having a proximal end and a distal end;
a branch that connects to the distal end of the trunk;
wherein each of the trunk and the branch comprises a tube of one or more tissue engineered sheets having a lumen; and
an aperture on the trunk close to the distal end of the trunk and above the connection between the branch and the trunk.

3. A kit, comprising:
a tissue construct of claim 2; and
a second branch that is contralateral to, and separate from, the tissue construct;
wherein the second branch comprises a tube of one or more tissue engineered sheets having a lumen.

4. A kit of artificial tissue, comprising:
a trunk; and
two branches that are separate from each other and from the trunk;
wherein each of the trunk and the branches comprises a tube of one or more tissue engineered sheets having a lumen.

5. An implant, comprising:
a trunk having a proximal end and a distal end;
one or two branches that connect to the distal end of the trunk;
wherein each of the trunk and the branches comprises a tube of one or more tissue engineered sheets having a lumen; and
one or more stents that are embedded within, mounted inside, of the sheets of one or more of the trunk and the one or two branches.

6. An implant, comprising:
a trunk having a proximal end and a distal end;
one or two branches that connect to the distal end of the trunk;
a tube of one or more tissue engineered sheets having a lumen disposed at the proximal end of the trunk; and
one or more sleeves of synthetic material disposed over the remainder of the trunks and the branches.

7. (canceled)

8. (canceled)

9. The implant of claim 5, wherein one or more of the stents is continuous or segmented.

10. The implant of claim 5, wherein one or more of the stents is balloon-expandable, self-expandable, collapsible and re-expandable, or adjustable.

11. The implant of claim 5, wherein one or more of the stents or part of the stents is resorbable.

12. The implant of claim 5, wherein one or more of the stents comprises a series of barbs.

13. The implant of claim 5, further comprising:
a synthetic support sleeve inside or outside the sheets of one or more of the trunk and the one or two branches.

14. An implant, comprising:
a trunk having a proximal end and a distal end, wherein the trunk comprises a stent, and a tube of one or more tissue engineered sheets disposed on an exterior surface of the stent at the proximal end of the trunk.

15. (canceled)

16. The implant of claim 14, further comprising a sheath of synthetic material disposed on an exterior surface of the stent at the distal end of the trunk, and abutting the tube of tissue.

17. The implant of claim 14, wherein the tube of tissue comprises cells harvested from an allogeneic, autologous, or xenogeneic source.

18. The implant of claim 5, further comprising one or more fenestrations.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. The implant of claim 6, further comprising one or more fenestrations.

46. The implant of claim 14, further comprising one or more fenestrations.
